(19) United States
(12) Patent Application Publication
(10) Pub. No.: US 2013/0344009 A1
(43) Pub. Date: Dec. 26, 2013

(54) FOAMING ORAL CARE FORMULATION AND SYSTEM AND METHOD OF FORMING AND USING SAME

(71) Applicant: Children Oral Care, LLC, Buena Park, CA (US)

(72) Inventor: Puneet Nanda, Cerritos, VA (US)

(21) Appl. No.: 13/925,749

(22) Filed: Jun. 24, 2013

Related U.S. Application Data
(60) Provisional application No. 61/663,454, filed on Jun. 22, 2012.

(51) Int. Cl. A61K 8/04; A61Q 11/00

(52) U.S. Cl. CPC A61K 8/046; A61Q 11/00
USPC 424/49

(57) ABSTRACT

Oral care compositions, systems, and methods of using and forming the compositions and systems are disclosed. The composition includes a surfactant and/or a foaming agent to cause the composition to foam prior to administration to an oral cavity. The foaming composition readily and quickly reaches interstitial areas within the oral cavity and at a gum line. In addition, the foaming action makes the composition enjoyable for use by children, which, in turn, encourages compliance with use.

### CROSS REFERENCE TO RELATED APPLICATION

This application claims the benefit and priority of Provisional Application No. 61/663,454, filed on Jun. 22, 2012, entitled FOAMING ORAL CARE FORMULATION AND SYSTEM, AND METHOD OF FORMING AND USING SAME, the contents of which are hereby incorporated by reference to the extent the contents do not conflict with the present disclosure.

### FIELD OF THE INVENTION

The present disclosure generally relates to oral care compositions and methods. More particularly, the disclosure relates to foaming oral care compositions and methods for promoting and maintaining oral health.

### BACKGROUND OF THE INVENTION

Maintaining oral health is generally important for a variety of reasons. If oral health is not maintained, a variety of conditions, ranging from bad breath to general health problems, may occur. Such conditions include, for example, tooth decay, tooth coloration, gum disease, tooth loss, and even general health problems, such as heart disease, stroke, poorly controlled diabetes and preterm labor. Unfortunately, poor oral health affects millions of people every year.

The presence of plaque in an oral cavity can lead to such oral and general health problems. Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing with toothpaste and flossing the affected areas. If not removed, however, the presence of plaque can give rise to tartar formation, tooth decay, gingivitis, periodontitis, and other health problems.

Typical methods of brushing with toothpaste include applying a desired amount of toothpaste onto a brush, optionally applying water to the toothpaste, brushing, and rinsing the oral cavity with water when the brushing is complete. Although such techniques work relatively well, they require a water source for rinsing and generally require facilities for the water source and for a place to spit out the toothpaste and water. In addition, the toothpaste may be relatively thick and/or viscous and therefore resist getting into small spaces between teeth and/or between a tooth and a gum line. Also, typical toothpastes may not be desirable to or suitable for young children. Accordingly, improved formulations, systems, and methods for maintaining and improving oral health are desired.

### SUMMARY OF THE INVENTION

The present disclosure provides a foaming oral care composition, a system including the composition, and methods of forming and using the composition. The ways in which the present disclosure addresses various drawbacks of now-known oral care compositions are discussed in greater detail below. However, in general, use of the composition, system, and method, in accordance with various embodiments of the disclosure, assists in preventing and/or mitigating plaque build-up (e.g., especially interstitially and along a gum line), as well as reducing the effects of the same. Additionally and/or alternatively, the composition may be used to reduce bad breath (malodor). The foaming composition and system of the present disclosure can be used without the requirement of a water source and allow the composition to be applied directly to an oral cavity. Because the composition foams, the composition can reach areas within an oral cavity (e.g., between teeth, at the gum line, and on a tongue surface) that might not otherwise be reached with typical toothpastes. Further, in accordance with exemplary embodiments of the disclosure, the composition is suitable for young children, and, in accordance with further embodiments, the composition and method of using the composition are designed to be enjoyable for children to use to thereby encourage use by children.

In accordance with various exemplary embodiments of the present disclosure, a composition includes an effective amount of one or more active ingredients, such as anticaries, at least one surfactant or foaming agent to facilitate forming a foaming composition, and at least one solvent, such as water, and the composition foams when dispensed from a pump-actuated container. Additionally or alternatively, the one or more active ingredients include enzymes, such as naturally occurring protolytic and non-protolytic enzymes that reduce inflammation, help reduce or prevent plaque buildup, whiten teeth, reduce bacteria, and/or promote a healthy flora within an oral cavity. In this case, the composition may be buffered to facilitate stable and efficacious delivery of the one or more active ingredients. In accordance with additional aspects of the disclosure, the composition optionally includes one or more cleaning and/or whitening agents. In accordance with additional aspects of the disclosure, the composition optionally includes one or more moisturizing agents. In accordance with additional aspects of the disclosure, the composition optionally includes one or more preservatives. In accordance with additional aspects of the disclosure, the composition optionally includes one or more sweeteners. In accordance with additional aspects of the disclosure, the composition optionally includes one or more thickening agents. The composition may also optionally include one or more flavorants and/or colorants. The composition may also include abrasive material, such as colloidal or hydrated silica, which may be in suspension. The composition may include any combination of these and other optional ingredients. An exemplary composition may comprise, consist essentially of, or consist of one or more active ingredients, at least one surfactant or foaming agent, at least one solvent, and any combination of the optional ingredients set forth herein.

In accordance with further embodiments of the disclosure, a system for maintaining and improving oral health includes a composition including an effective amount of one or more active ingredients (e.g., anticaries and/or enzymes), at least one surfactant, and at least one solvent and a foam-actuating (e.g., propellant-free) container, which causes the composition to foam as it is dispensed from the container. Use of a propellant-free composition is less expensive to manufacture, does not expose the user to hydrocarbon propellants, and typically provides a superior consumer experience, compared to propellant-based oral care products. In accordance with exemplary aspects of these embodiments, the container includes a propellant-free pump actuator coupled to a vial (e.g., a non-resilient container) and the container/vial is configured to mix air with the composition to form a foam or foaming composition. In accordance with further aspects of these embodiments, the composition optionally includes one or more cleaning and/or whitening agents, one or more moisturizing agents, one or more preservatives, one or more sweeteners, one or more thickening agents, one or more flavorants and/or colorants, one or more abrasive substances (e.g., colloidal silica), or any combination thereof.

In accordance with additional embodiments of the disclosure, a method of using the composition or system (e.g., any composition or system described herein) includes the steps of forming a foaming composition using a pump actuator apparatus (e.g., propellant-free) and applying a dose of the foaming composition to a portion of an oral cavity. Because the composition foams prior to application to a surface within an oral cavity and pre-disperses one or more active ingredients within the composition, the composition can quickly reach interstitial and other areas within the cavity and can deliver the active ingredients much quicker to treatment areas within the oral cavity. In accordance with various aspects of these embodiments, the foaming composition can be applied directly to an oral cavity. Alternatively, the composition may be applied to a utensil, such as a toothbrush or tongue brush, prior to application to an oral cavity.

In accordance with yet additional embodiments of the disclosure, a method of forming the composition includes the step of admixing an effective amount of one or more active ingredients, such as anticaries and/or enzymes, at least one surfactant to facilitate forming a foaming composition, and at least one solvent, such as water. In accordance with additional aspects of these embodiments, the method optionally additionally includes admixing one or more cleaning and/or whitening agents, one or more moisturizing agents, one or more preservatives, one or more sweeteners, one or more thickening agents, and/or one or more flavorants and/or colorants to a mixture including the one or more active ingredients.

### DETAILED DESCRIPTION OF EXEMPLARY EMBODIMENTS

The present disclosure provides an improved oral care composition and system and methods of forming and using the composition and system. As set forth in greater detail below, the oral care composition and system, in accordance with various embodiments of the disclosure, can be used when a water source is not available and provide active ingredients to an oral cavity in a foaming composition, which facilitates distribution of the composition to otherwise difficult-to-reach places within the oral cavity. Moreover, use of exemplary systems and compositions were found to be enjoyable by children (e.g., under the age of 13), which was found to encourage use of the oral care composition and system.

In accordance with various embodiments of the disclosure, a composition includes an effective amount of one or more active agents (e.g., anticaries and/or enzymes), at least one surfactant or foaming agent, and at least one solvent. The at least one surfactant and the at least one solvent work together to cause the composition to foam—e.g., when activated by a pump-actuated container—e.g., a propellant-free system. The foaming action may be desirable because it facilitates relatively rapid and controlled application of a relatively small amount of an otherwise low viscosity composition to desired areas—e.g., within an oral cavity. In addition, the foaming liquid may facilitate increased speed and efficacy of the composition for a given amount of active agent.

Exemplary anticaries agents include sodium fluoride, stannous fluoride, and sodium monofluorophosphate.

Exemplary enzymes include whitening and anti-inflammatory, and bactericidal enzymes, such as lysozyme, glucoxidase, amylase, papain, amyloguoside, peptizyme, and lactoferrin. Exemplary compositions include two or more enzymes selected from the group consisting of: lysozyme, peptizyme, and papain. In accordance with a particular aspect, the composition includes lysozyme, peptizyme, and papain. Alternatively, the one or more enzymes may include two or more enzymes selected from the group consisting of lysozyme, amylase, amyloglucosidase, glucosidase, peptizyme, and papain. In accordance with a particular aspect, the composition includes lysozyme, amylase, amyloglucosidase, glucoxidase, peptizyme, and papain.

In accordance with further exemplary embodiments, a composition includes a metal ion management formulation. An exemplary metal ion management formulation includes one or more compounds that bind with iron and other metal ions to inhibit growth of gram negative bacteria. Exemplary compounds suitable for inhibiting growth of gram negative bacteria include sodium EDTA, phytic acid, lactoferrin, and various combinations thereof.

Exemplary surfactants/foaming agents include nonionic surfactants, such a polyol surfactant having a molecular weight approximately 12,500, sold under the name Pluronic F 127 by BASF, cocamidoproyl betaine—e.g., concentrated cocamidoproyl betaine sold under the name Tego® by Goldschmidt AG, and the like. Use of concentrated cocamidoproyl betaine may be particularly desirable because it facilitates suspension of abrasive particles, such as colloidal silica, when such is included in the suspension.

Exemplary solvents include water (e.g., purified water), propylene glycol, and the like.

In accordance with various aspects of these embodiments, the composition may further include one or more cleaning and/or whitening agents, moisturizing agents, preservative, sweeteners, flavorants, colorants, additional actives, additional surfactants, thickening agents, pH modifiers, and any combination of these compounds.

Exemplary cleaning/whitening agents include sodium hexametaphosphate, hydrogen peroxide, carbamide peroxide and the like.

Exemplary moisturizing agents include Aloe Vera extract and the like.

Exemplary preservatives include sodium benzoate and the like.

Exemplary sweeteners include sugar alcohols, such as xylitol, artificial sweeteners, such as sucralose, and the like.

Exemplary flavorants include natural and artificial flavors, such as bubble gum flavor, peppermint, menthol and the like.

Exemplary colorants include any suitable coloring agent, including FD&C Blue 1.

Exemplary abrasives include colloidal and/or hydrated particles (e.g., silica). Colloidal particles may have an average diameter of about 5 to about 500 nm, or about 5 to about 100 nm, or about 5 to about 50 nm. Hydrated particles may have an average diameter ranging from about 5 to about 25 microns. Surprisingly and unexpectedly, compositions including such abrasive particles can be used with, foaming pump actuators as described herein. Typical abrasive particles will clog most foaming actuators and are therefore not suitable for use with the same, but colloidal/hydrated particles (e.g., silica), such as those having the size ranges described herein are suitable for pump foam actuators.

Exemplary additional active ingredients include any active ingredients that facilitate oral health, such as actives configured to reduce plaque and/or provide a source of calcium. By way of example, zinc gluconate and/or calcium lactate may be an additional active ingredient.

Exemplary thickening agents include glycerin, silicon oxide (e.g., hydrated silica, sold by American International Chemical, Inc.), xanthan gum, and the like.

Exemplary pH modifiers and buffering agents include acidic (e.g., citric acid) and basic compounds.

An exemplary system includes a non-resilient container and a pump foam actuator. An exemplary container and actuator are disclosed in U.S. Pat. No. 5,443,569, issued on Aug. 22, 1995, in the name of Uehira et al. Use of a system with a pump-foam actuator allows direct application of the composition to desired areas within the oral cavity and other advantages as noted herein.

### SPECIFIC EXAMPLES

The following non-limiting examples illustrate exemplary compositions in accordance with various embodiments of the disclosure. The examples are merely illustrative; it is not intended that the disclosure be limited to the examples. Exemplary compositions in accordance with the present disclosure may include the ingredients listed below as well as additional and/or alternative inert materials, preservatives, and other constituents typically found in oral rinse compositions. Where exemplary inert materials, preservatives, and/or the like are listed, these ingredients are merely exemplary, and it is understood that other similar ingredients may be substituted for the materials listed in the examples below.

### Example 1

A foaming oral rinse composition, including the ingredients listed below, is formed by admixing the listed ingredients.

| | | | Exemplary |
|---|---|---|---|
| | | FORMULA | Range |
| INGREDIENT | USE | (w/w %) | (w/w %) |
| PURIFIED WATER | SOLVENT | 62.65% | 50-70 |
| XYLITOL | SWEETENER | 16.00% | 10-30 |
| SODIUM BENZOATE | PRESERVATIVE | 0.30% | 0.01-1 |
| COCAMIDOPROYL | SURFACTANT/ | 3.00% | 1-5 |
| BETAIN | FOAMING AGENT | | |
| SUCRALOSE | SWEETENER | 0.10% | 0.01-1 |
| GLYCERIN | SOLVENT | 15.00% | 5-20 |
| CITRIC ACID | PH MODIFIER | 0.004% | 0-1 |
| PLURONIC F 127 | SURFACTANT/ | 2.00% | 0.5-5 |
| | FOAMING AGENT | | |
| SODIUM | CLEANING/ | 0.50% | 0-2 |
| HEXAMETA- | WHITENING | | |
| PHOSPHATE | AGENT | | |
| BUBBLE GUM | FLAVOR | 0.30% | 0-1 |
| FLAVOR | | | |
| ALOE VERA | MOISTURIZER | 0.100% | 0-1 |
| EXTRACT | | | |
| SODIUM FLUORIDE | ANTI CAVITY | 0.05% | 0.01-0.25 |
| FD&C BLUE 1 | COLOR | 0.00% | 0-.5 |
| HYDRATED SILICA | ABRASIVE | 0.00% | 0-2 |
| TOTAL | | 100.000% | |

The composition of example 1 effectively reduced an amount of plague on a surface in an oral cavity. And, the composition was found enjoyable to use by children under 13.

### Example 2

A foaming oral rinse composition, including the ingredients listed below, is formed by transferring the formula amount of purified water to a mixing tank, setting the impeller speed to 40 Hz, and turning the mixer on. The formula amount of the sodium fluoride is then added to the tank and mixed for about 15 minutes. The formula amounts of sodium benzoate, citric acid, sucralose, sodium hexametaphosphate, Aloe Vera extract, xylitol, and tetrasodium pyrophosphate are added and mixed for about 30 minutes. The formula amount of Pluronic F127 is then added and mixed with the batch for about 45 minutes. In a separate container, 12% of the glycerin and the formula amounts of xanthan gum and hydrated silica are mixed together for about 45 minutes—until the solids are dissolved. The glycerin mixture and the remaining formula amount of glycerin are then added to the batch and mixed for about 30 minutes. The Tegobetain F50 is then added to the batch and mixed for about 15 minutes. The mixture is then allowed to settle.

In a separate container, the formula amount of menthol is melted and the bubble gum flavor is added to the menthol and mixed in a mixer for about 15 minutes. The menthol mixture is then transferred to the batch and mixed for about 30 minutes. The mixture is then allowed to settle before quality assurance testing and packaging.

| | | | Exemplary |
|---|---|---|---|
| | | FORMULA | Range |
| INGREDIENT | USE | (w/w %) | (w/w %) |
| PURIFIED | SOLVENT | 58.53% | 50-70 |
| WATER | | | |
| XYLITOL | SWEETENER | 25.00% | 10-30 |
| SODIUM | PRESERVATIVE | 0.30% | 0.01-1 |
| BENZOATE | | | |
| TEGOBETAIN F | SURFACTANT/ | 2.00% | 1-5 |
| 50 | FOAMING AGENT | | |
| SUCRALOSE | SWEETENER | 0.16% | 0.01-1 |
| GLYCERIN | SOLVENT | 10.00% | 5-20 |
| CITRIC ACID | PH MODIFIER | 0.100% | 0-1 |
| PLURONIC F | SURFACTANT/ | 0.90% | 0.5-5 |
| 127 | FOAMING AGENT | | |
| SODIUM | CLEANING/ | 0.50% | 0-5 or 0-2 |
| HEXAMETA- | WHITENING | | |
| PHOSPHATE | AGENT | | |
| BUBBLE GUM | FLAVOR | 0.10% | 0-1 |
| FLAVOR | | | |
| MENTHOL | FLAVOR | 0.01% | 0-1 |
| XANTHAM GUM | THICKENING | 0.005% | 0-.1 |
| | AGENT | | |
| TETRASODIUM | TARTAR | 2.00% | 1-5 |
| PYROPHOSPATE | CONTROL | | |
| | AGENT | | |
| ALOE VERA | MOISTURIZER | 0.100% | 0-1 |
| EXTRACT | | | |
| SODIUM | ANTI CAVITY | 0.243% | 0.01-0.3 |
| FLUORIDE | AGENT | | |
| HYDRATED | THICKENER/ | 0.05% | 0-10, 0.01-10 |
| SILICA | ABRASIVE | | or 0.01-0.1 |
| COLLOIDAL | ABRASIVE | | 0-10 |
| SILICA | | | |
| TOTAL | | 100.000% | |

The composition of example 2 effectively reduced an amount of plague on a surface in an oral cavity, and the composition was found enjoyable to use by children under 13.

### Example 3

A foaming oral rinse composition, including the ingredients listed below, is formed by transferring the formula amount of purified water to a mixing tank, setting the impeller speed to 40 Hz, and turning the mixer on. While mixing and recirculating the water, the formula amount of the sodium fluoride is then added to the tank and mixed for about 20 minutes until complete dissolution. While continuing to mix and recirculate, the formula amounts of sodium benzoate, citric acid, sucralose, sodium hexametaphosphate, Aloe Vera extract, xylitol, and tetrasodium pyrophosphate are added and mixed for about 30 minutes until complete dissolution. The formula amount of Pluronic F127 is then added and mixed with the batch for about 45 minutes until complete dissolution. In a separate container, 10% of the glycerin and the formula amounts of xanthan gum are mixed together for about 15 minutes—until well dispersed. While continuing to mix the glycerin mixture, the hydrated silica is added and mixed for about 30 minutes—until well dispersed. The glycerin mixture and the remaining formula amount of glycerin are then added to the batch and mixed for about 45 minutes. The Tegobetain F50 is then added to the batch and mixed for about 15 minutes. The mixture is then allowed to settle.

In a separate container, the formula amount of menthol is heated to about 50° C. to about 60° C. Once melted, the menthol is removed from the heat and the bubble gum flavor is added to the menthol and mixed in a mixer for about 15 minutes. The menthol mixture is then transferred to the batch and mixed for about 30-45 minutes. The mixture is then allowed to settle before quality assurance testing and packaging.

| | | | Exemplary |
|---|---|---|---|
| | | FORMULA | Range |
| INGREDIENT | USE | (w/w %) | (w/w %) |
| PURIFIED | SOLVENT | 57.8900% | 50-70 |
| WATER | | | |
| XYLITOL | SWEETENER | 25.0000% | 10-30 |
| SODIUM | PRESERVATIVE | 0.3000% | 0.01-1 |
| BENZOATE | | | |
| TEGOBETAIN F | SURFACTANT/ | 2.0000% | 1-5 |
| 50 | FOAMING AGENT | | |
| SUCRALOSE | SWEETENER | 0.2800% | 0.01-1 |
| GLYCERIN | SOLVENT | 10.0000% | 5-20 |
| CITRIC ACID | PH MODIFIER | 0.6215% | 0-1 |
| PLURONIC F | SURFACTANT/ | 0.9000% | 0.5-5 |
| 127 | FOAMING AGENT | | |
| SODIUM | CLEANING/ | 0.5000% | 0-5 or 0-2 |
| HEXAMETA- | WHITENING | | |
| PHOSPHATE | AGENT | | |
| BUBBLE GUM | FLAVOR | 0.1000% | 0-1 |
| FLAVOR | | | |
| MENTHOL | FLAVOR | 0.0100% | 0-1 |
| XANTHAM GUM | THICKENING | 0.0050% | 0-.1 |
| | AGENT | | |
| TETRASODIUM | TARTAR | 2.0000% | 1-5 |
| PYROPHOSPATE | CONTROL | | |
| | AGENT | | |
| ALOE VERA | MOISTURIZER | 0.1000% | 0-1 |
| EXTRACT | | | |
| SODIUM | ANTI CAVITY | 0.2430% | 0.01-0.3 |
| FLUORIDE | AGENT | | |
| HYDRATED | THICKENER/ | 0.05% | 0- 10, 0.01-10 |
| SILICA | ABRASIVE | | or 0.01-0.1 |
| COLLOIDAL | ABRASIVE | | 0-10 |
| SILICA | | | |
| TOTAL | | 100.000% | |

The composition of example 3 effectively reduced an amount of plague on a surface in an oral cavity. In addition, children under the age of 13 found the composition to be enjoyable to use.

For examples 1-3, the pH is between about 5 and 7; the specific gravity is between about 1 and 1.5 g/cc; the sodium fluoride concentration is between about 0.2187 and 0.2673 wt %; the appearance is clear to slightly hazy and colorless.

### Example 4

A foaming oral rinse composition, including the ingredients listed below, is formed by transferring the formula amount of purified water to a mixing tank, setting the impeller speed to 30 Hz, and turning the mixer on. The formula amounts of Aloe Vera, zinc gluconate, benzoic acid, sodium phosphate, and phytic acid are added to the mixer and mixed for about 30 minutes. The formula weight of xylitol is added to the mixture and mixed for about 30 minutes. The formula amount of Pluronic F-127 is added to the mixture and the mixture is mixed for at least 120 minutes. The formula amount of sorbitol is then added and mixed for about 15 minutes. In a separate container, about 14% of the total propanediol is added to a container and mixed with the formula amounts of cellulose gum and the enzyme pre-blend and mixed for at least 45 minutes. The remaining propanediol is added to the water mixture and mixed for at least 30 minutes. The propanediol mixture (including the enzyme mixture) is then added to the water mixture and mixed for at least 20 minutes. In a separate container, the formula amount of menthol is melted and natural peppermint flavor is added to the melted menthol and mixed. This mixture is then added to the water mixture and mixed for about 15 minutes. The mixture is then allowed to settle and test samples are taken.

The composition of example 4 can be used as a tongue cleaner. In this case, the composition can be applied directly to a tongue or to a tongue cleaning device, such as a brush or a scraper. Use of the composition of example 4 was found to reduce malodor within or emanating from an oral cavity, and to provide a fresh, clean feeling within the oral cavity.

The mixture of this example has a colorless to off-white color. The appearance is clear to hazy. The pH is 3.5-5. And, the specific gravity is between about 1 and 1.1 g/cc.

| | | | Exemplary |
|---|---|---|---|
| | | FORMULA | Range |
| INGREDIENT | USE | (w/w %) | (w/w %) |
| PURIFIED | SOLVENT | 64.957% | 50-70 |
| WATER | | | |
| ALOE VERA | MOISTURIZER | 0.0500 | 0-0.1 |
| XYLITOL | SWEETENER | 10.000% | 5-20 |
| BENZOIC ACID | PRESERVATIVE | 0.0100% | 0.001-1 |
| ZINC | ACTIVE | 0.0200% | 0.001-0.1 |
| GLUCONATE | | | |
| SORBITOL | SWEETENER/ | 12.75% | 5-20 |
| | SUGAR ALCOHOL | | |
| DISODIUM | | 0.01 | 0.001-0.1 |
| PHOSPHATE | | | |
| PHYTIC ACID | METAL ION | 0.0001 | 0-0.1 |
| | MANAGEMENT | | |
| PLURONIC F | SURFACTANT/ | 3.000% | 0.5-5 |
| 127 | FOAMING AGENT | | |
| PEPPERMINT | FLAVOR | 0.0150% | 0-1 |
| FLAVOR | | | |
| MENTHOL | FLAVOR | 0.0380% | 0-1 |
| CELLULOSE GUM | THICKENING | 0.050% | 0-0.1 |
| | AGENT | | |
| PROPANEDIOL | SOLVENT | 8.8500% | 1-15 |
| MULTI-ENZYME | ACTIVES/ | 0.25% | 0.01-1 |
| PRE-BLEND | ENZYMES | | |
| TOTAL | | 100.000% | |

There are several unmet needs that are not fulfilled by current oral care products that are fulfilled by the tooth-foam composition set forth herein. For example, children generally do not like brushing their teeth and a significant issue parents identify with proper oral care is developing proper habits and getting children to brush properly. In addition, parents wish that it was more fun for children to brush their teeth.

The tooth-foam composition of the present disclosure has been highly rated by children in studies. Exemplary techniques of using tooth-foam compositions are similar to spraying whip cream in mouth, which is an activity children enjoy. Studies have shown that children (e.g., 13 and under) enjoy using the tooth-foam composition of the present disclosure.

In addition, the tooth foam composition more effectively reaches hard to reach places to provide superior oral care based on its liquid, rather than paste consistency. Further, the tooth-foam composition of the present disclosure creates less mess when used, compared to typical toothpastes.

Although exemplary embodiments of the present invention are set forth herein, it should be appreciated that the invention is not so limited. Various modifications, variations, and enhancements in composition and method set forth herein may be made without departing from the spirit and scope of the present invention.

I claim:

1. A foaming oral care composition comprising:
an effective amount of one or more anticaries agents;
at least one surfactant; and
at least one solvent,
wherein the composition foams when activated by a propellant-free pump actuator coupled to a container.

2. The foaming oral care composition of claim 1, further comprising one or more cleaning agents.

3. The foaming oral care composition of claim 1, further comprising a whitening agent.

4. The foaming oral care composition of claim 1, further comprising a moisturizing agent.

5. The foaming oral care composition of claim 1, further comprising a preservative.

6. The foaming oral care composition of claim 1, further comprising a sweetener.

7. The foaming oral care composition of claim 1, further comprising a flavorant.

8. The foaming oral care composition of claim 1, further comprising a second surfactant.

9. The foaming oral care composition of claim 1, further comprising a thickening agent.

10. The foaming oral care composition of claim 1, further comprising abrasive material.

11. The foaming oral care composition of claim 10, wherein the abrasive material comprises colloidal silica.

12. The foaming oral care composition of claim 10, wherein the abrasive material is in suspension.

13. A foaming oral care system comprising a pump-actuated container and a composition according to claim 1.

14. The foaming oral care system of claim 13, wherein the composition further comprising abrasive material.

15. The foaming oral care system of claim 14, wherein the abrasive material comprises colloidal silica.

16. The foaming oral care composition of claim 14, wherein the abrasive material is in suspension.

17. A method of oral care, the method comprising the step of administering a composition according to claim 1 directly to a portion of an oral cavity.

18. The method of oral care of claim 17, comprising a step of using a pump-actuated container to administer the composition directly to a portion of the oral cavity.

19. The method of oral care of claim 17, wherein the composition further comprises abrasive material.

20. The method of oral care of claim 17, wherein the abrasive material comprises colloidal silica.
